(19) United States
(12) Patent Application Publication
(10) Pub. No.: US 2003/0220300 A1
(43) Pub. Date: Nov. 27, 2003

(54) Reduction of hair growth

(76) Inventors: Cheng Shine Hwang, Framingham, MA (US); Gurpreet S. Ahluwalia, Potomac, MD (US); Anwar Jardien, Brookline, MA (US); Douglas Shander, Acton, MA (US)

Correspondence Address: FISH & RICHARDSON PC, 225 FRANKLIN ST, BOSTON, MA, 02110, US

(21) Appl. No.: 10/145,283

(22) Filed: May 14, 2002

(51) Int. Cl. A61K 31/675; A61K 31/426

(52) U.S. Cl. 514/91; 514/369

(57) ABSTRACT

Mammalian hair growth can be reduced by topical application of a thiazolidinone.

### BACKGROUND

The invention relates to reducing hair growth in mammals, particularly for cosmetic purposes.

A main function of mammalian hair is to provide environmental protection. However, that function has largely been lost in humans, in whom hair is kept or removed from various parts of the body essentially for cosmetic reasons. For example, it is generally preferred to have hair on the scalp but not on the face.

Various procedures have been employed to remove unwanted hair, including shaving, electrolysis, depilatory creams or lotions, waxing, plucking, and therapeutic antiandrogens. These conventional procedures generally have drawbacks associated with them. Shaving, for instance, can cause nicks and cuts, and can leave a perception of an increase in the rate of hair regrowth. Shaving also can leave an undesirable stubble. Electrolysis, on the other hand, can keep a treated area free of hair for prolonged periods of time, but can be expensive, painful, and sometimes leaves scarring. Depilatory creams, though very effective, typically are not recommended for frequent use due to their high irritancy potential. Waxing and plucking can cause pain, discomfort, and poor removal of short hair. Finally, antiandrogens—which have been used to treat female hirsutism—can have unwanted side effects.

It has previously been disclosed that the rate and character of hair growth can be altered by applying to the skin inhibitors of certain enzymes. These inhibitors include inhibitors of 5-alpha reductase, omithine decarboxylase, S-adenosylmethionine decarboxylase, gamma-glutamyl transpeptidase, and transglutaminase. See, for example, Breuer et al., U.S. Pat. No. 4,885,289; Shander, U.S. Pat. No. 4,720,489; Ahluwalia, U.S. Pat. No. 5,095,007; Ahluwalia et al., U.S. Pat. No. 5,096,911; and Shander et al., U.S. Pat. No. 5,132,293.

Thiazolidinediones are a class of therapeutic drugs used for the treatment of type II diabetes also known as non-insulin-dependent diabetes mellitus (NIDDM) (R. R. Henry (1997), current therapies for diabetes, 26(3), p.553-573, J. A. B. Balfour et al. (1999), Drugs, 57(6), 921-930, J. M. Lawrence et al. (2000), international journal of clinical practice, 54(9), 614, 618). Thiazolidinediones reduce serum glucose levels in type II diabetic patients through increasing insulin sensitivity in liver, adipose tissue and muscle. Clinical data have shown that administration of thiazolidinediones increase glucose uptake, oxidation and storage in muscle and lipolysis in adipose tissue, and reduces glucose production and secretion in liver. Moreover, thiazolidinediones can decrease serum free fatty acid (FFA) and triglyceride levels and increase high-density lipoprotein-cholesterol levels in patients with hyperlipidemia. Therefore, thiazolidinediones is indicated to be of potential use for the treatment of other diseases including, hypertension and cardiovascular disease.

Recent studies indicate that thiazolidinediones can inhibit in vitro proliferation of cancer cell and keratinocytes (R. M. Moretti et al. (2001), Int. J. Cancer, 92, p.733-737. And C. N. Ellis et al. (2000) Arch. Dermatol, 136, p. 609-616.) However, studies from Breider et al. indicate that administration of thiazolidinediones can increase proliferation of cardiac endothelial cell and brown adipocytes in vivo (M. A. Breider et al. (1999), Toxicology Pathology, 27(5), p.545-552).

Thiazolidinediones may regulate cellular functions by multiple mechanisms, since they have diverse biological effects. One mechanism through which thiazolidinediones are believed of to have biological effect is their ability to serve as a ligand for peroxisome proliferator-activated receptor gamma (PPARγ). PPAR family is a subset of nuclear receptor superfamily, which includes steroid and non-steroid hormone receptors. Nuclear receptors are transcription factors that activate gene expression upon interaction with the ligands. Ligand-bound nuclear receptors can regulate expression of genes, which are involved in various biological functions (J. C. Corton et al. (2000) Annu. Rev. Pharmacol. Toxicol., 40, p.491-518.). PPAR family contains three members: PPARα, PPARδ and PPAR-γ. PPARs, activated by natural or synthetic ligands, bind to the PPAR response element in the promoter region of target genes and subsequently increase expression of target genes. Each member of PPAR family performs different physiological functions, based on their divergent patterns of tissue-specific expression, different ligand-binding specificities and divergent physiological consequences when activated. PPARα is mainly involved in hepatic lipid metabolism and PPARγ is mainly involved in adipocyte differentiation and metabolism.

### SUMMARY

In one aspect, the invention provides a method (typically a cosmetic method) of reducing unwanted mammalian (preferably human) hair growth by applying to the skin a thiazolidinone derivative in an amount effective to reduce hair growth. The unwanted hair growth may be undesirable from a cosmetic standpoint or may result, for example, from a disease or an abnormal condition (e.g., hirsutism).

Preferred thiazolidinone derivates have the formula:

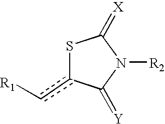

where X and Y are, independently, oxygen, nitrogen, or sulfur, with the proviso that at least X or Y is an oxygen; R₁ is an aryl group; and R₂ is hydrogen or —(CH₂)ₙA, wherein n=1 to 5 and A=CO₂H, PO₃H or SO₃H.

For example, X and Y may both be oxygen, X may be sulfur and Y oxygen, or X may be oxygen and Y nitrogen; R₂ may be hydrogen or —(CH₂)ₙCO₂H, wherein n=1 to 5; and R₁ may have the formula:

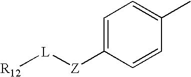

where Z is oxygen or a carbonyl linkage L is (CH₂), or R₁₂₀N(CH₂)ₙ, where n=1 to 4, and R₁₂₀ is an alkyl, alkoxy, alkylcarbonyl or arylalkyl group having from 1 to 12 carbon atoms; and R₁₂ has the formula:

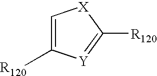

where X is, independently oxygen, nitrogen or sulfur and Y is nitrogen; and where R₁₂₀ is an alkyl, alkoxy, alkylcarbonyl or arylalkyl group having from 1 to 12 carbon atoms.

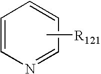

where R₁₂₁ is hydrogen or an alkyl group having from 1 to 4 carbon atoms;

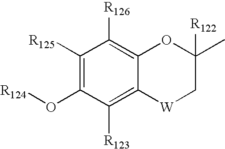

where R₁₂₂ and R₁₂₃ are, independently, hydrogen or an alkyl group having from 1 to 5 carbon atom; R₁₂₄ is hydrogen, an aliphatic acyl group having from 1 to 6 carbon atoms, an alicyclic acyl group having from 1 to 6 carbon atoms, a heterocyclic acyl group having from 1 to 6 carbon atoms, an aromatic acyl group having from 7 to 14 carbon atoms, an araliphatic acyl group having from 7 to 14 carbon atoms, an alkoxycarbonyl group having from 2 to 7 carbon atoms, an araalkyloxycarbonyl group having from 8 to 16 carbon atoms; and R₁₂₅ and R₁₂₆ are (a) independently, an alkyl group or alkoxy group having from 1 to 5 carbon atom; or (b) together a alkylenedioxy group having from 1 to 4 carbon atom; or

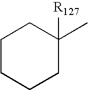

where R₁₂₇ is hydrogen or an alkyl group having from 1 to 4 carbon atoms; or R₁ may have the formula

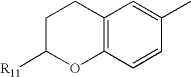

where R11 is a benzyl group; or R1 may have the formula

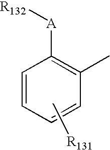

where A is oxygen, sulfur, SO, SO₂ CH₂O, or CH₂S, and R₁₃₁, and R₁₃₂ independently are hydrogen, a halogen, an alkyl group having from 1 to 4 carbon atoms, an aryl group having from 6 to 12 carbon atoms, hydroxy, an alkoxy group having from 1 to 6 carbon atoms, an aryloxy group having from 6 to 12 carbon atoms, an aryloxy group having from 6 to 12 carbon atoms, cyano, nitro, an alkylcarbamido group having from 1 to 6 carbon atoms, an arylcarbamido group having from 7 to 14 carbon atoms, a dialkylcarbamido group having from 2 to 8 carbon atoms, a diarylcarbamide having from 13 to 26 carbon atoms, an alkylarylcarbamido having from 7 to 14 carbon atoms, an alkylthiocarbamido group having from 1 to 6 carbon atoms, an arylthiocarbamido group having from 7 to 14 carbon atoms, a dialkylthiocarbamido group having from 3 to 8 carbon atoms, an diarylthiocarbamido group having from 13 to 26 carbon atoms, an alkylarylthiocarbamido group having from 8 to 16 carbon atoms, amino, an alkylamino group having from 1 to 6 carbon atoms, an arylamino group having from 6 to 12 carbon atoms, an dialkylamino group having from 2 to 12 carbon atoms, a diarylamino group having from 12 to 24 carbon atoms, an arylalkylamino group having from 7 to 15 carbon atoms, an aminocarbonyl group an alkylaminocarbonyl group having from 2 to 8 carbon atoms, an arylaminocarbonyl group having from 7 to 14 carbon atoms, a dialkylaminocarbonyl group having from 3 to 11 carbon atoms, a diarylaminocarbonyl group having from 13 to 22 carbon atoms, an arylalkylaminocarbonyl group having from 8 to 15 carbon atoms, an alkylcarbonyloxy group having from 2 to 8 carbon atoms, an arylcarbonyloxy group having from 7 to 14 carbon atoms, a carboxyl, an alkoxycarbonyl group having from 2 to 8 carbon atoms, an aryloxycarbonyl group having from 7 to 15 carbon atoms, sulfo, an alkylsulfonylamido group having from 1 to 6 carbon atoms, an arylsulfonylamido group having from 6 to 12 carbon atoms, an alkylsulfonyl group having from 1 to 6 carbon atoms, an arylsulfonyl group having from 6 to 12 carbon atoms, an alkylsulfinyl group having from 1 to 6 carbon atoms, an arylsulfinyl group having from 6 to 12 carbon atoms, or a heteroaryl group having from 2 to 6 carbon atoms.

Typically, in practicing the aforementioned method, the thiazolidinone derivative will be included in a topical composition along with a dermatologically or cosmetically acceptable vehicle. Accordingly, the present invention also relates to topical compositions comprising a dermatologically or cosmetically acceptable vehicle and a thiazolidinone derivative in an amount effective to reduce hair growth.

In addition, the present invention relates to the use of a thiazolidinone derivative for the manufacture of a therapeutic topical composition for reducing hair growth.

In another aspect, the invention provides a method (typically a cosmetic method) of reducing unwanted mammalian hair growth by applying to the skin a ligand of PPARγ in an amount effective to reduce hair growth.

Other features and advantages of the invention may be apparent from the description of the preferred embodiments thereof, and from the claims.

### DETAILED DESCRIPTION

An example of a preferred composition includes at least one thiazolidinone derivative in a cosmetically and/or dermatologically acceptable vehicle. The composition may be a solid, semi-solid, or liquid. The composition may be, for example, a cosmetic and dermatologic product in the form of an, for example, ointment, lotion, foam, cream, gel, or solution. The composition may also be in the form of a shaving preparation or an aftershave. Examples of preferred thiazolidinone derivatives are these having the formula described above. Specific examples are ciglitazone, pioglitazone, rosiglitazone, troglitazone, and 5-(5-nitro-2-phenylsulfanyl-benzylidene)-2-thioxo-thiazolidin-4-one. These compounds are known.

The composition may include more than one thiazolidinone derivative. In addition, the composition may include one or more other types of hair growth reducing agents, such as those described in U.S. Pat. Nos. 4,885,289; 4,720,489; 5,132,293; 5,096,911; 5,095,007; 5,143,925; 5,328,686; 5,440,090; 5,364,885; 5,411,991; 5,648,394; 5,468,476; 5,475,763; 5,554,608; 5,674,477; 5,728,736; 5,652,273; WO 94/27586; WO 94/27563; and WO 98/03149, all of which are incorporated herein by reference.

The concentration of the ligand in the composition may be varied over a wide range up to a saturated solution, preferably from 0.1% to 30% by weight or even more; the reduction of hair growth increases as the amount of ligand applied increases per unit area of skin. The maximum amount effectively applied is limited only by the rate at which the ligand penetrates the skin. The effective amounts may range, for example, from 10 to 3000 micrograms or more per square centimeter of skin.

The vehicle can be inert or can possess cosmetic, physiological and/or pharmaceutical benefits of its own. Vehicles can be formulated with liquid or solid emollients, solvents, thickeners, humectants and/or powders. Emollients include stearyl alcohol, mink oil, cetyl alcohol, oleyl alcohol, isopropyl laurate, polyethylene glycol, petroleum jelly, and myristyl myristate. Solvents include ethyl alcohol, isopropanol, acetone, diethylene glycol, ethylene glycol, dimethyl sulfoxide, and dimethyl formamide.

The composition also can include components that enhance the penetration of the inhibitor into the skin and/or to the site of action. Examples of penetration enhancers include urea, polyoxyethylene ethers (e.g., Brij-30 and Laureth-4), 3-hydroxy-3,7,11-trimethyl-1,6,10-dodecatriene, terpenes, cis-fatty acids (e.g., oleic acid, palmitoleic acid), acetone, laurocapram, dimethylsulfoxide, 2-pyrrolidone, oleyl alcohol, glyceryl-3-stearate, propan-2-ol, myristic acid isopropyl ester, cholesterol, and propylene glycol. A penetration enhancer can be added, for example, at concentrations of 0.1% to 20% or 0.5% to 5% by weight.

The composition also can be formulated to provide a reservoir within or on the surface of the skin to provide for a continual slow release of the ligand. The composition also may be formulated to evaporate slowly from the skin, allowing the thiazolidinone derivative extra time to penetrate the skin.

### EXAMPLE 1

A composition prepared containing 10% by weight of rosiglitazone in a vehicle containing 80% ethanol, 17.5% water, 2% propylene glycol dipelargonate (Emerest 2388), and 0.5% propylene glycol. Using this example a 40% reduction in hair mass was detected after three weeks in the Golden Syrian Hamster assay discussed later.

### EXAMPLE 2

A composition prepared containing 1.3% by weight of ciglitazone in a vehicle containing 80% ethanol, 17.5% water, 2% propylene glycol dipelargonate (Emerest 2388), and 0.5% propylene glycol. Using this example a 22.7% reduction in hair mass was detected after three weeks in the Golden Syrian Hamster assay.

### EXAMPLE 3

A composition prepared containing 5% by weight of pioglitazone in a vehicle containing 80% ethanol, and 20% dimethyl sulfoxide. Using this example a 47.7% reduction in hair mass was detected after three weeks in the Golden Syrian Hamster assay.

### EXAMPLE 4

A composition of the aforementioned compound (in Example 3) in a vehicle containing 80% ethanol, 17.5% water, 2% propylene glycol dipelargonate (Emerest 2388), and 0.5% propylene glycol.

The composition should be topically applied to a selected area of the body from which it is described to reduce hair growth. For example, the composition can be applied to the face, particularly to the beard area of the face, i.e., the cheek, neck, upper lip, and chin. The composition also may be used as an adjunct to other methods of hair removal including shaving, waxing, mechanical epilation, chemical depilation, electrolysis and laser-assisted hair removal.

The composition can also be applied to the legs, arms, torso or armpits. The composition is particularly suitable for reducing the growth of unwanted hair in women having hirsutism or other conditions. In humans, the composition should be applied once or twice a day, or even more frequently, to achieve a perceived reduction in hair growth. Perception of reduced hair growth could occur as early as 24 hours or 48 hours (for instance, between normal shaving intervals) following use or could take up to, for example, three months. Reduction in hair growth is demonstrated when, for example, the rate of hair growth is slowed, the need for removal is reduced, the subject perceives less hair on the treated site, or quantitatively, when the weight of hair removed (i.e., hair mass) is reduced.

Golden Syrian Hamster Assay

Male intact Golden Syrian hamsters are considered acceptable models for human beard hair growth in that they display oval shaped flank organs, one on each side, each about 8 mm. in major diameter. These organs produce fine light colored hair typical of the animal pelage found on the body. In response to androgens the flank organs produce dark coarse hair similar to male human beard hair. To evaluate the effectiveness of a composition in reducing hair growth, the flank organs of each of a group of hamsters are depilated by applying a thioglycolate based chemical depilatory (Surgex) and/or shaved. To one organ of each animal 10 μl. of vehicle alone once a day is applied, while to the other organ of each animal an equal amount of vehicle containing the ligand under evaluation is applied. After three weeks of topical applications (one application per day for five days a week), the flank organs are shaved and the amount of recovered hair (hair mass) from each is weighed. Percent-reduction of hair growth is calculated by subtracting the hair mass (mg) value of the test compound treated side from the hair mass value of the vehicle treated side; the delta value obtained is then divided by the hair mass value of the vehicle treated side, and the resultant number is multiplied by 100.

The above-described assay will be referred to herein as the “Golden Syrian hamster” assay or “hair mass” assay. Preferred compositions provide a reduction in hair growth of at least about 15% and more preferably at least about 35%, when tested in the Golden Syrian hamster assay.

Human Hair Follicle Growth Assay

Tissue source—Human skin was obtained from a plastic surgeon as a by-product of face-lift procedures. Immediately after removal, the skin was placed in Williams E medium containing antibiotics and refrigerated. The Williams E medium is a commercially obtained medium which has been formulated with essential nutrients for maintaining viability of tissues or cells such as of hair follicle in an in-vitro environment.

Hair Follicle Isolation and Culture—Human hair follicles in growth phase (anagen) were isolated from face-lift tissue (obtained from plastic surgeons) under dissecting scope using a scalpel and watchmakers forceps. The skin was sliced into thin strips exposing 2-3 rows of follicles that could readily be dissected. Follicles were placed into 0.5 ml William's E medium (Life Technologies, Gaithersburg, Md.) supplemented with 2 mM L-glutamine, 10 μg/ml insulin, 10 ng/ml hydrocortisone, 100 units of penicillin, 0.1 mg/ml streptomycin and 0.25 μg/ml amphotericin B. The follicles were incubated in 24-well plates (1 follicle/well) at 37° C. in an atmosphere of 5% CO₂ and 95% air. Thiazolidinediones are dissolved into dimethyl sulfoxide as 100-fold stock solution. The control hair follicles were treated with dimethyl sulfoxide without thiazolidinedione. The follicles were photographed in the 24-well plates under the dissecting scope at a power of 10×. Typically, image recordings were made on day 0 (day follicles were placed in culture), and again on day 7. The length of Hair follicle was assessed using an image analysis software system (Jasc Image Robot). The growth of hair fiber was calculated by the subtracting the follicle length on day 0 from that determined on day 7.

Results

The data from hamster hair mass assay indicate that the thiazolidinediones ciglitazone, pioglitazone and rosiglitazone can reduce hair growth in vivo as shown in Table I. The data from human hair follicle growth assay indicate that the thiazolidinediones ciglitazone, pioglitazone, troglitazone, rosiglitazone and 5-(5-nitro-2-phenylsulfanyl-benzylidene)-2-thioxo-thiazolidin-4-one can reduce human hair growth as shown in Table II. Moreover, the reduction of hair growth by thiazolidinones is dose-dependent as shown in Tables III, IV, V and VI.

TABLE I
Reduction of hamster hair mass by thiazolidinediones

| Compound | Dose (%) | Vehicle* | Treated (mg) | Control (mg) | % Reduction |
|---|---|---|---|---|---|
| Ciglitazone | 1.3 | A | 2.78 ± 0.23 | 3.37 ± 0.35 | 22.7 ± 5.8 |
| Pioglitazone | 5 | B | 1.32 ± 0.06 | 2.64 ± 0.24 | 47.7 ± 4.1 |
| Rosiglitazone | 10 | A | 1.78 ± 0.71 | 3.03 ± 0.2 | 40.0 ± 5.0 |

*Vehicles:
A - 80% ethanol, 17.5% water, 2% propylene glycol dipelargonate (Emerest 2388), and 0.5% propylene glycol.
B - 80% ethanol, 20% dimethyl sulfoxide

TABLE II
Reduction of human hair follicle growth by thiazolidinones
Hair follicle length increase (mm)

| Compound | Dose (μM) | Treated | Control | % Reduction |
|---|---|---|---|---|
| Ciglitazone | 20 | 0.19 ± 0.24 | 1.54 ± 0.6 | 87.5 ± 15.6 |
| Pioglitazone | 50 | 0.15 ± 0.07 | 1.13 ± 0.39 | 86.7 ± 6.2 |
| Rosiglitazone | 100 | 0.24 ± 0.16 | 0.97 ± 0.17 | 75.2 ± 16.5 |
| Troglitazone | 100 | 0.11 ± 0.08 | 1.29 ± 0.14 | 91.5 ± 6.2 |
| 5-(5-Nitro-2- | 10 | 0.11 ± 0.08 | 2.04 ± 0.19 | 94.6 ± 3.9 |
| phenylsulfanyl- | | | | |
| benzylidene)-2- | | | | |
| thioxo- | | | | |
| thiazolidin-4-one | | | | |

TABLE III
Dose-dependent reduction of human hair follicle growth by ciglitazone

| Ciglitazone (μM) | Hair follicle length increase (mm) | % Reduction |
|---|---|---|
| 0 | 2.17 ± 0.67 | 0 |
| 3 | 1.34 ± 0.5 | 38.2 ± 23.0 |
| 6 | 0.54 ± 0.3 | 75.1 ± 13.8 |
| 9 | 0.2 ± 0.2 | 90.8 ± 9.2 |

TABLE IV
Dose-dependent reduction of human hair follicle growth by pioglitazone

| Pioglitazone (μM) | Hair follicle length increase (mm) | % Reduction |
|---|---|---|
| 0 | 0.96 ± 0.3 | 0 |
| 15 | 0.67 ± 0.19 | 30.2 ± 19.8 |
| 30 | 0.23 ± 0.16 | 76.0 ± 16.7 |
| 45 | 0.1 ± 0.14 | 89.6 ± 14.6 |

TABLE V
Dose-dependent reduction of human hair follicle by rosiglitazone

| Rosiglitazone (μM) | Hair follicle length increase (mm) | % Reduction |
|---|---|---|
| 0 | 1.41 ± 0.26 | 0 |
| 20 | 0.92 ± 0.29 | 34.8 ± 20.6 |
| 40 | 0.74 ± 0.15 | 47.5 ± 10.6 |
| 60 | 0.52 ± 0.2 | 63.1 ± 14.1 |

TABLE VI
Dose-dependent reduction of human hair follicle growth by
5-(5-Nitro-2-phenylsulfanyl-benzylidene)-2-thioxo-thiazolidin-4-one

| 5-(5-Nitro-2- | | |
|---|---|---|
| phenylsulfanyl- | | |
| benzylidene)-2- | | |
| thioxo-thiazolidin-4- | | |
| one (μM) | Hair follicle length increase (mm) | % Reduction |
| 0 Control | 1.12 ± 0.22 | 0 |
| 0.1 | 0.37 ± 0.17 | 66.9 ± 15.2 |
| 0.5 | 0.16 ± 0.17 | 85.7 ± 15.2 |
| 1.0 | 0.07 ± 0.08 | 93.7 ± 7.1 |

Other embodiments are within the claims.

What is claimed is:

1. A method of reducing mammalian hair growth which comprises selecting an area of skin from which reduced hair growth is desired; and
applying to said area of skin a dermatologically acceptable composition comprising a thiazolidinone derivative in an amount effective to reduce hair growth.

2. The method of claim 1, wherein the thiazolidinone derivative is a compound having the following formula: 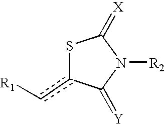
wherein X and Y are, independently, oxygen nitrogen, or sulfur, with the proviso that at least X or Y is an oxygen; R₁ is an aryl group; and R₂ is H or (CH₂)ₙA, wherein n is 1 to 5 and A is CO₂H, or SO₃H.

3. The method of claim 2, wherein X and Y are oxygens.

4. The method of claim 2, wherein X is sulfur and Y is oxygen.

5. The method of claim 2, wherein X is oxygen and Y is nitrogen.

6. The method of claims 2-5, wherein R₂ is hydrogen or —(CH₂)ₙA, wherein n is 1 to 5 and A is CO₂H, PO₃H or SO₃H.

7. The method of claim 6, wherein R₁ has the formula 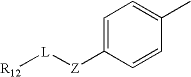
where Z is oxygen or a carbonyl linkage L is (CH₂)ₙ or R₁₂₀N(CH₂)ₙ, where n=1 to 4, and R₁₂₀ is an alkyl, alkoxy, alkylcarbonyl or arylalkyl group having from 1 to 12 carbon atom; and R₁₂₀ has the formula 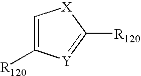
where X is, independently oxygen, nitrogen or sulfur and Y is nitrogen; and where R₁₂₀ is an alkyl, alkoxy, alkylcarbonyl or arylalkyl group having from 1 to 12 carbon atoms;
where R₁₂₁ is hydrogen or an alkyl group having from 1 to 4 carbon atoms; 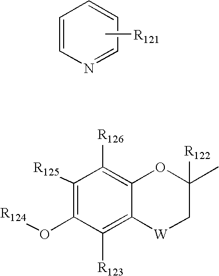
where R₁₂₂ and R₁₂₃ are, independently, hydrogen or an alkyl group having from 1 to 5 carbon atom; R₁₂₄ is hydrogen, an aliphatic acyl group having from 1 to 6 carbon atoms, an alicyclic acyl group having from 1 to 6 carbon atoms, a heterocyclic acyl group having from 1 to 6 carbon atoms, an aromatic acyl group having from 2 to 8 carbon atoms, an araliphatic acyl group having from 7 to 14 carbon atoms, an alkoxycarbonyl group having from 2 to 7 carbon atoms, or an araalkyloxycarbonyl group having from 8 to 16 carbon atoms; and R₁₂₅ and R₁₂₆ are (a) independently, an alkyl group or alkoxy group having from 1 to 5 carbon atom; or (b) together a alkylenedioxy group having from 1 to 4 carbon atom; or 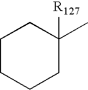
where R₁₂₇ is hydrogen or an alkyl group having from 1 to 4 carbon atoms.

8. The method of claim 6, wherein R₁ has the formula: 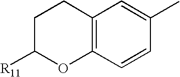
where R₁₁ is a benzyl group.

9. The method of claim 6, wherein R₁ has the formula: 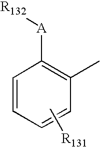
where A is oxygen, sulfur, SO, SO₂ CH₂O, or CH₂S, and R₁₃₁, and R₁₃₂ independently are hydrogen, a halogen, an alkyl group having from 1 to 4 carbon atoms, an aryl group having from 6 to 12 carbon atoms, hydroxy, an alkoxy group having from 1 to 6 carbon atoms, an aryloxy group having from 6 to 12 carbon atoms, an aryloxy group having from 6 to 12 carbon atoms, cyano, nitro, an alkylcarbamido group having from 1 to 6 carbon atoms, an arylcarbamido group having from 7 to 14 carbon atoms, a dialkylcarbamido group having from 2 to 8 carbon atoms, a diarylcarbamide having from 13 to 26 carbon atoms, an alkylarylcarbamido having from 7 to 14 carbon atoms, an alkylthiocarbamido group having from 1 to 6 carbon atoms, an arylthiocarbamido group having from 7 to 14 carbon atoms, a dialkylthiocarbamido group having from 3 to 8 carbon atoms, an diarylthiocarbamido group having from 13 to 26 carbon atoms, an alkylarylthiocarbamido group having from 8 to 16 carbon atoms, amino, an alkylamino group having from 1 to 16 carbon atoms, an arylamino group having from 6 to 12 carbon atoms, an dialkylamino group having from 2 to 12 carbon atoms, a diarylamino group having from 12 to 24 carbon atoms, an arylalkylamino group having from 7 to 15 carbon atoms, an aminocarbonyl group, an alkylaminocarbonyl group having from 2 to 8 carbon atoms, an arylaminocarbonyl group having from 7 to 14 carbon atoms, a dialkylaminocarbonyl group having from 3 to 11 carbon atoms, a diarylaminocarbonyl group having from 13 to 22 carbon atoms, an arylalkylaminocarbonyl group having from 8 to 15 carbon atoms, an alkylcarbonyloxy group having from 2 to 8 carbon atoms, an arylcarbonyloxy group having from 7 to 14 carbon atoms, a carboxyl, an alkoxycarbonyl group having from 2 to 8 carbon atoms, an aryloxycarbonyl group having from 7 to 15 carbon atoms, sulfo, an alkylsulfonylamido group having from 1 to 6 carbon atoms, an arylsulfonylamido group having from 6 to 12 carbon atoms, an alkylsulfonyl group having from 1 to 6 carbon atoms, an arylsulfonyl group having from 6 to 12 carbon atoms, an alkylsulfinyl group having from 1 to 6 carbon atoms, an arylsulfinyl group having from 6 to 12 carbon atoms, or a heteroaryl group having from 2 to 6 carbon atoms.

10. The method of claim 1, wherein the thiazolidinone derivative is ciglitazone.

11. The method of claim 1, wherein the thiazolidinone derivative is pioglitazone.

12. The method of claim 1, wherein the thiazolidinone derivative is rosiglitazone.

13. The method of claim 1, wherein the thiazolidinone derivative is troglitazone.

14. The method of claim 1, wherein the thiazolidinone derivative is darglitazone.

15. The method of claim 1, wherein the thiazolidinone derivative is englitazone.

16. The method of claim 1, wherein the thiazolidinone derivative is 5-(5-nitro-2-phenylsulfanyl-benzylidene)-2-thioxo-thiazolidin-4-one.

17. The method of claim 1, wherein the concentration of said thiazolidinone derivative in said composition is between 0.1% and 30%.

18. The method of claim 1, wherein the composition provides a reduction in hair growth of at least 15% when tested in the Golden Syrian Hamster assay.

19. The method of claim 1, wherein the composition provides a reduction in hair growth of at least 35% when tested in the Golden Syrian Hamster assay.

20. The method of claim 1, wherein the thiazolidinone derivative is applied to the skin in an amount of from 10 to 3000 micrograms of said compound per square centimeter of skin.

21. The method of claim 1, wherein said mammal is a human.

22. The method of claim 21, wherein said area of skin is on the face of a human.

23. The method of claim 22, wherein the composition is applied to the area of skin in conjunction with shaving.

24. The method of claim 21, wherein said area of skin is on a leg of the human.

25. The method of claim 21, wherein said area of skin is on an arm of the human.

26. The method of claim 21, wherein said area of skin is in an armpit of the human.

27. The method of claim 21, wherein said area of skin is on the torso of the human.

28. The method of claim 1, wherein the composition is applied to an area of skin of a woman with hirsutism.

29. The method of claim 1, wherein said hair growth comprises androgen stimulated hair growth.

30. The method of claim 1, wherein the composition further includes a second compound that also causes a reduction in hair growth.

31. The method of claim 1 where the thiazolidinone derivative has enantiomeric excess of the active isomer.

32. A method of reducing mammalian hair growth which comprises selecting an area of skin from which reduced hair growth is desired, and
applying to said area of skin a dermatologically acceptable composition comprising a ligand of peroxisome proliferator-activated receptor gamma PPARγ, in an amount effective to reduce hair growth.
